(19) United States
(12) Patent Application Publication
(10) Pub. No.: US 2002/0082215 A1
(43) Pub. Date: Jun. 27, 2002

(54) Method for treating ischemic events affecting the central nervous system

(75) Inventor: William H. Frey II, White Bear Lake, MN (US)

Correspondence Address: Chiron Corporation, Intellectual Property Department, P.O. Box 8097, Emeryville, CA, 94662-8097, US

(73) Assignee: Chiron Corporation

(21) Appl. No.: 09/976,620

(22) Filed: Oct. 12, 2001

Related U.S. Application Data
(60) Provisional application No. 60/240,549, filed on Oct. 14, 2000.

(51) Int. Cl. A61K 38/18

(52) U.S. Cl. 514/12

(57) ABSTRACT

The present invention is directed methods for reducing or preventing ischemic damage in the central nervous system of a mammal. The methods comprise administering to the nasal cavity of the mammal a pharmaceutical composition comprising a therapeutically effective amount of IGF-I or biologically active variant thereof. The IGF-I or variant thereof is absorbed through the nasal cavity and transported into the central nervous system of the mammal in an amount effective to reduce or prevent ischemic damage associated with an ischemic event. The methods are useful in treating a mammal that has experienced an ischemic event or that is at risk of experiencing such an event.

### CROSS-REFERENCE TO RELATED APPLICATION

This application claims the benefit of U.S. Provisional Application Serial No. 60/240,549, filed Oct. 14, 2000, the content of which is herein incorporated by reference in its entirety.

### FIELD OF THE INVENTION

The present invention is directed to methods for treatment of ischemic events affecting the central nervous system in a mammal, more particularly to intranasal administration of insulin-like growth factor-I (IGF-I) to reduce or prevent ischemic damage in the central nervous system in association with an ischemic event.

### BACKGROUND OF THE INVENTION

Neurotrophic factors such as insulin-like growth factor-I (IGF-I) regulate the survival and differentiation of nerve cells during the development of the peripheral and central nervous systems. In the mature nervous system, neurotrophic factors maintain the morphologic and neurochemical characteristics of nerve cells and strengthen functionally active synaptic connections.

The potential neuroprotective effects of insulin-like growth-1 (IGF-I) have been investigated in animal models of focal and global ischemia, where IGF-I has been administered either systemically (e.g, intraperitoneally) or by intracerebroventricular injection (Gluckman et al. (1992) Biochem. Biophys. Res. Comm. 182:593-599; Tagami et al. (1997) Lab. Invest. 76:613-617; Zhu and Auer (1994) J. Cereb. Blood Flow Metab. 14:237-242; Guan et al. (1993) J. Cereb. Blood Flow Metab. 13:609-616; Guan et al. (1996) Neuro. Report 7:632-636; Johnston et al. (1996) J. Clin. Invest. 97:300-308); Guan et al. (2000) J. Cereb. Bloodflow Metals 20:513-519; Loddick et al. (1998) Proc. Natl. Acad. Sci. USA 95:1894-1898; U.S. Pat. No. 5,714,460). Other evidence in support of IGF-I as a potential therapeutic agent for injury to the central nervous system comes from studies utilizing tissue culture (Gluckman et al. (1992) Biochem. Biophys. Res. Comm. 182:593-599; Cheng and Mattson (1992) J. Neurosci. 12:1558-1566; U.S. Pat. No. 5,652,214).

Since IGF-I does not cross the blood-brain barrier efficiently, developing a noninvasive method for delivering IGF-I to the central nervous system (CNS) is important. Intracerebroventricular (ICV) administration is effective but not practical for the large number of individuals who require treatment for ischemic events affecting the CNS. Intranasal (IN) delivery is a promising, non-invasive and practical new method of bypassing the blood-brain barrier to deliver IGF-I to the CNS (U.S. Pat. No. 5,624,898). Previous work has demonstrated that IGF-I can be delivered to the CNS, including the brain and spinal cord, directly from the nasal cavity following IN administration, achieving brain concentrations in the nanomolar range (Thorne et al. (1999) Growth Hormone and IGFRes. 9:387; International Publication No. WO 00/33813). Recent studies demonstrate that IN delivery of IGF-I to the CNS occurs along both the olfactory and trigeminal neural pathways (Thome et al. (2000) Society for Neuroscience Abstracts 26:1365, Abstract #511.18; International Publication No. WO 00/33813). Intranasal delivery of low dose (less than 0.10 mg per kg body weight) IGF-I and administration of low dose IGF-I to tissues innervated by the trigeminal nerve have been recognized as having potential therapeutic benefit for treatment of stroke and other central nervous system disorders (International Publication Nos. WO 00/33813 and WO 00/33814).

Delivery of IGF-I to the brain could be a promising strategy for the treatment of ischemic events affecting the CNS in a mammal. However, it has yet to be demonstrated that intranasally delivered IGF-I achieves biologically active concentrations that are sufficient to protect against ischemic damage in animal models of ischemic events such as stroke. The present invention is based upon the discovery that a beneficial therapeutic effect is achieved with intranasal administration of IGF-I following experimentally induced focal cerebral ischemia and reperfusion.

### SUMMARY OF THE INVENTION

Methods for reducing or preventing ischemic damage in the central nervous system of a mammal are provided. The methods comprise administering intranasally (IN) a therapeutically effective amount or dose of IGF-I, or biologically active variant thereof, to the mammal's nasal cavity, preferably to the upper one-third of the nasal cavity. The IGF-I or biologically active variant thereof can then be absorbed through a mucosa or epithelium and transported to the central nervous system of the mammal, by way of a neural pathway and in an amount effective for reducing or preventing ischemic damage in the central nervous system (CNS) of the mammal. Intranasal administration of the therapeutically effective amount of IGF-I or biologically active variant thereof in accordance with the methods of the present invention provides a noninvasive means of delivering this neuroprotective agent to the CNS in an amount that is effective at reducing ischemic damage in a mammal that has experienced an ischemic event or preventing ischemic damage in a mammal at risk of experiencing an ischemic event. The methods find use in the treatment of a mammal prior to or following an ischemic event that affects the CNS.

### BRIEF DESCRIPTION OF THE DRAWINGS

FIG. 1 shows the effect of intranasally administered (IN) IGF-I on corrected infarct volume (*p=0.001 compared with control-1, +p=0.004 compared with control-2 and with the 37.5 μg dose).

FIG. 2 shows the effect of IN IGF-I on motor-sensory function in the postural reflex and hemiparesis test (*p=0.02, **p=0.01 compared with control-2 and with the 37.5 μg dose; # p=0.027 compared with control-1).

FIG. 3 shows the effect of IN IGF-I on sensorimotor performance in the left forepaw placing test (*p=0.008 compared with control-2 and with the 37.5 μg dose).

FIG. 4 shows the effect of IN IGF-I on vestibulomotor function in the beam balance test (*=0.005 compared with the 37.5 μg dose; # p=0.001 compared with the 37.5 μg dose and with control-2).

FIG. 5 shows body weight loss after the onset of MCAO.

FIG. 6 shows the effect of IN IGF-I on infarct size. Infarct sizes with treatment beginning at 2 hours or 4 hours following MCAO in IN IGF-I treated groups were lower than that of the respective controls (*p=0.02, # p=0.04).

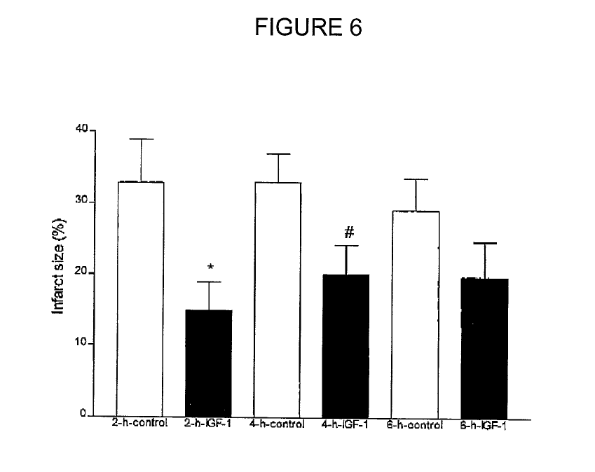

FIG. 7 shows the effect of IN IGF-I on motor-sensory function assessed by the postural reflex test. The change of deficit scores over time was significantly different between the IGF-I and control groups (p=0.007), and the deficit scores were borderline significantly lower than the scores in the control at the 5, 6, and 7 day points (*p≦0.05) in rats treated initially at 2 hours following MCAO. The change over time was not significantly different between the IN IGF-I and control groups in rats treated initially at 4 hours and 6 hours following MCAO.

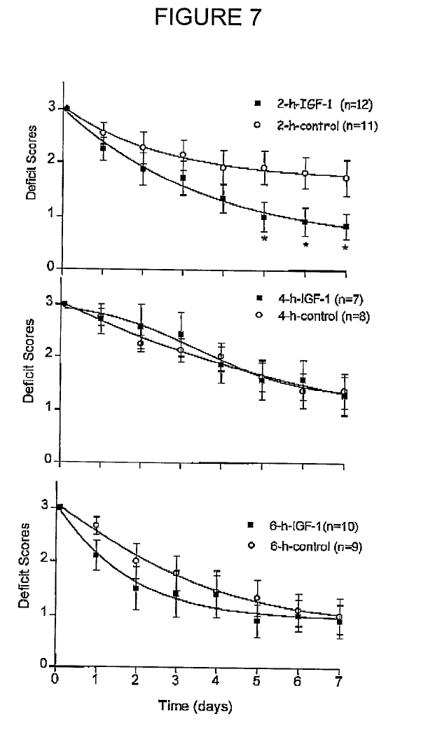

FIG. 8 shows the effect of IN IGF-I on somatosensory function assessed by the contact time of the adhesive tape test. The change of deficit scores over time was significantly different between the IGF-I and the control groups in rats treated initially at 2 hours following MCAO (*p=0.02), but not significantly different in rats treated initially at 4 hours and 6 hours following MCAO. There was no significant difference between IGF-1 and control groups at the 1-7 day time points in rats where treatment was initiated at 2 hours, 4 hours, or 6 hours following MCAO.

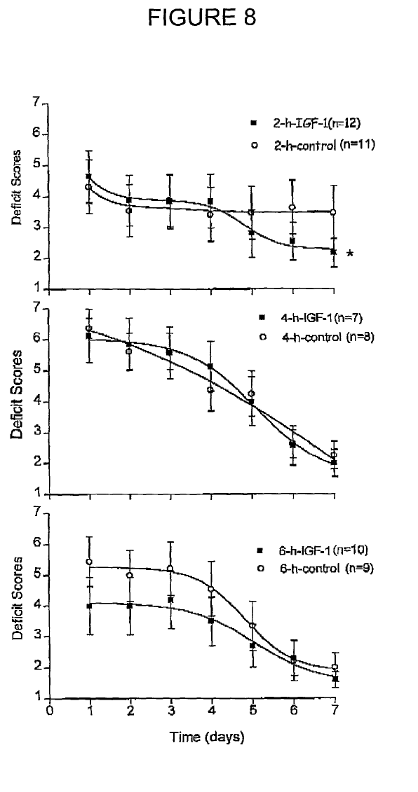

FIG. 9 shows the effect of IGF-I on somatosensory function assessed by the removal time of the adhesive tape test. The change of deficit scores over time was significantly different between the IN IGF-I and control groups in rats treated initially at 6 hours following MCAO (*p=0.04), but not significantly different in rats treated initially at 2 hours or 4 hours following MCAO. There was no significant difference between the IGF-I and control groups at the 1-7 day time points in rats where treatment was initiated at 2 hours, 4 hours, or 6 hours following MCAO.

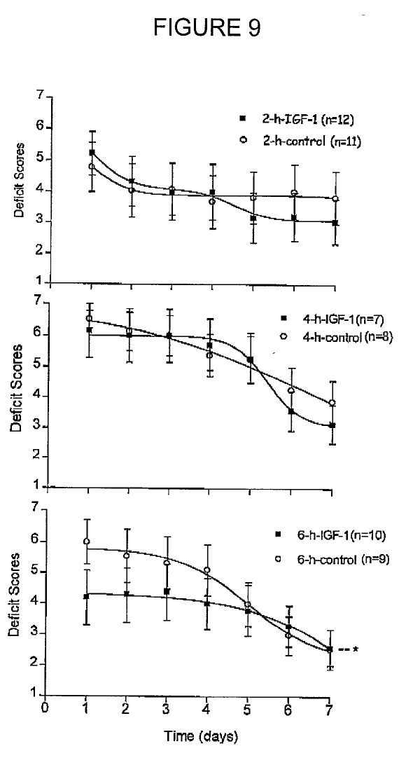

FIG. 10 shows the effect of IGF-I on vestibulomotor function assessed by the beam balance test. There was a significant change in deficit scores over time, but this change was not significantly different between the IGF-I and the control groups.

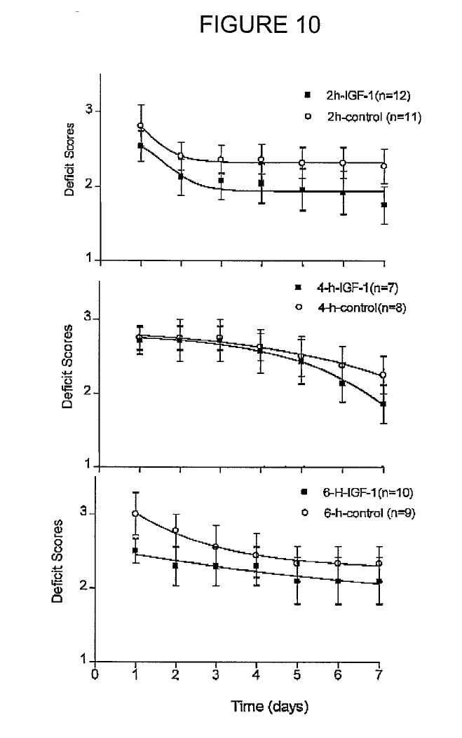

### DETAILED DESCRIPTION OF THE INVENTION

The present invention is directed to methods for reducing or preventing ischemic damage in the central nervous system (CNS) of a mammal. The methods comprise administering intranasally (IN) a therapeutically effective amount of insulin-like growth factor-I (IGF-I) or biologically active variant thereof to the CNS of the mammal. The methods find use in treating a mammal following an ischemic event, whereby ischemic damage is reduced, or for treating a mammal that is at risk of experiencing an ischemic event, whereby ischemic damage is prevented if the ischemic event occurs.

By “reducing” is intended the decreasing, slowing, or ameliorating of the ischemic damage. By “preventing” is intended the putting off, delaying, inhibiting, or otherwise stopping the onset of the ischemic damage. Ischemic damage for the purposes of the present invention refers to damage to the tissues of the CNS, including the brain and/or spinal cord, as a result of ischemia.

By “ischemia” is intended a condition within the CNS that results from a deficient supply of blood to the cells within the tissues of the CNS. Ischemia can involve, for example, restricted blow flow to the brain or spinal cord as a result of blockage of a single artery that normally supplies these tissues (i.e., focal ischemia), or may involve a general restriction of blood flow to the entire brain, forebrain, or spinal cord (i.e., global ischemia). With focal ischemia, restricted blood flow through the single artery results in the death of all cellular elements (pan-necrosis) in the region of the CNS supplied by that artery. With global ischemia, certain vulnerable regions throughout the affected tissues exhibit cell death, particularly death of neurons.

For the purposes of the present invention, ischemic damage includes death of neuronal and glial cells, edema or swelling of the tissues in the affected areas of the CNS, and development of one or more neurologic deficits, including loss of motor, sensory, vestibulomotor, and/or somatosensory function. An infarction results when the blood supply to a localized area is deprived so that damage occurs to neuronal tissue. An “infarct” is an area of coagulation necrosis in a tissue resulting from obstruction of circulation to the area. Intranasal administration of a therapeutically effective amount or dose of IGF-I to the CNS is effective at reducing or preventing ischemic damage, including reducing infarct size, edema, and neurologic deficit, in a mammal that has experienced an ischemic event or that is at risk of experiencing an ischemic event.

Ischemia and the resulting ischemic damage in the CNS are brought on by an ischemic event. By “ischemic event” is intended any instance that results, or could result, in a deficient supply of blood to the tissues of the CNS, including the brain and/or spinal cord. Ischemic events encompassed by the present invention include, but are not limited to, stroke, such as stroke caused by emboli within cerebral vessels, arteriosclerotic vascular disease, the inflammatory processes, which frequently occur when thrombi form in the lumen of inflamed vessels, or hemmorage; multiple infarct dementia; cardiac failure and cardiac arrest; shock, including septic shock and cardiogenic shock; blood dyscrasias; hypotension; hypertension; an angioma; hypothermia; perinatal asphyxia; high altitude ischemia; hypertensive cerebral vascular disease; rupture of an aneurysm; seizure; bleeding from a tumor; and traumatic injury to the central nervous system, including open and closed head injury, neck injury, and spinal cord trauma such as occurs with a blow to the head, neck, or spine, or with an abrasion, puncture, incision, contusion, compression, and the like in any part of the head, neck, or vertebral column. Other possible ischemic events include traumatic injury due to constriction or compression of CNS tissue by, for example, subdural or intracranial hematoma, by a mass of abnormal tissue, such as a metastatic or primary tumor, by over accumulation of fluid, such as cerebrospinal fluid as a result of dysfunction of normal production, or by edema.

A mammal may be at risk of experiencing an ischemic event for medical or other reasons. For example, a mammal undergoing a cardiovascular surgical procedure, including, but not limited to, by-pass surgery, open-heart surgery, aneurysm surgery, and cardiac catheterization whether for treatment or diagnostic purposes may be at risk during or following the procedure. A mammal with a medical condition may be at risk of experiencing an ischemic event. Such medical conditions include, but are not limited to, herpes meningitis; hypertensive encephalopathy; myocardial infarction; and edema within a CNS tissue, such as results with viral infection or traumatic injuries noted above.

Intranasal administration of a therapeutically effective amount or dose of IGF-I or biologically active variant thereof in accordance with the methods of the present invention provides a method for treating a mammal that has experienced an ischemic event, or that is at risk of experiencing an ischemic event. By “treating” is intended the mammal experiencing the ischemic event, such as a stroke, or the mammal that is at risk of experiencing an ischemic event, incurs less focal ischemic damage and reduced neurologic deficits than would be observed in the absence of the treatment methods of the invention. Thus, for example, a mammal that has experienced a stroke and is undergoing IN IGF-I treatment in accordance with the methods of the present invention exhibits a reduction of ischemic damage, including reduction in infarct size, edema, and/or neurologic deficits (i.e., improved recovery of motor, sensory, vestibulomoter, and/or somatosensory function) beyond that seen without such treatment. Similarly, in a mammal at risk of experiencing an ischemic event, for example, a mammal undergoing a cardiovascular surgical procedure, IN IGF-I treatment prior to, during, and/or immediately following the surgical procedure in accordance with the methods of the present invention, ischemic damage can be prevented if the ischemic event occurs during or following the surgical procedure.

The methods of the invention comprise intranasal administration (IN) of a therapetically effective amount or dose of insulin-like growth factor-1 (IGF-I), or biologically active variant thereof, to the central nervous system (CNS) of a mammal that has ischemic damage as a result of an ischemic event, or that is at risk of having ischemic damage as a result of an ischemic event. By “intranasal administration” is intended the administering of IGF-I or biologically active variant thereof to a nasal cavity of the mammal, whereby this polypeptide is absorbed through the nasal cavity and transported into the central nervous system of the mammal by a neural pathway and in an amount effective for reducing or preventing ischemic damage as noted elsewhere herein. This method of administration allows for the noninvasive, direct delivery of IGF-I or biologically active variant thereof, also referred to as a neuroprotective agent herein, to the cerebral tissue that is affected by ischemia associated with the ischemic event, such as stroke, cardiac arrest, or other CNS injury as noted elswhere herein. Once delivered to the affected site, this neuroprotective agent reduces or prevents ischemic damage, including coagulation necrosis (i.e., infarct size or volume), edema, and/or neurologic deficit, relative to that seen for the mammal in the absence of treatment with intranasally administered IGF-I or biologically active variant thereof. As such, the methods of the invention elicit a therapeutic effect with regard to treating (i.e., reducing or preventing) ischemic damage associated with an ischemic event beyond that which occurs without intranasal administration of this neuroprotective agent.

More particularly, the methods of the invention administer IGF-I or biologically active variant thereof to the mammal of interest in a manner such that this neuroprotective agent is transported to the CNS, brain, and/or spinal cord along a neural pathway. A neural pathway includes transport within or along a neuron, through or by way of lymphatics running with a neuron, through or by way of a perivascular space of a blood vessel running with a neuron or neural pathway, through or by way of an adventitia of a blood vessel running with a neuron or neural pathway, or through an hemangiolymphatic system. The invention prefers transport of this neuroprotective agent by way of a neural pathway, rather than through the circulatory system, so that IGF-I, which is unable to, or only poorly, crosses the blood-brain barrier from the bloodstream into the brain, can be delivered to the CNS, brain, and/or spinal cord. In one embodiment, the IGF-I or biologically active variant thereof preferably accumulates in areas having the greatest density of receptor or binding sites for this neuroprotective agent.

Use of a neural pathway to transport a neurotrophic agent to the brain, spinal cord or other components of the central nervous system obviates the obstacle presented by the blood-brain barrier so that medications comprising IGF-I or variant thereof can be delivered directly to the CNS, more particularly to CNS tissues that have been damaged, or are at risk of damage, by the ischemic event. Although this neuroprotective agent once administered intranasally may be absorbed into the bloodstream as well as the neural pathway, preferably the IGF-I or variant thereof provides minimal effects systemically. In addition, intranasal administration in accordance with the method of the present invention can provide for delivery of a more concentrated level of IGF-I or variant thereof to CNS cells as this neuroprotective agent does not become diluted in fluids present in the bloodstream. As such, intranasal administration provides an improved method for delivering IGF-I or variant thereof to the CNS, brain, and/or spinal cord.

Thus, in one embodiment of the invention, intranasal administration includes delivery of IGF-I or biologically active variant thereof to the subject in a manner such that the neurotrophic agent is transported into the CNS, brain, and/or spinal cord along an olfactory neural pathway. Typically, such an embodiment includes administering this neuroprotective agent to tissue innervated by the olfactory nerve and inside the nasal cavity. Preferably, IGF-I or variant thereof is delivered to the olfactory area in the upper one-third of the nasal cavity and particularly to the olfactory epithelium.

Fibers of the olfactory nerve are unmyelinated axons of olfactory receptor cells that are located in the superior one-third of the nasal mucosa. The olfactory receptor cells are bipolar neurons with swellings covered by hair-like cilia that project into the nasal cavity. At the other end, axons from these cells collect into aggregates and enter the cranial cavity at the roof of the nose. Surrounded by a thin tube of pia, the olfactory nerves cross the subarachnoid space containing CSF and enter the inferior aspects of the olfactory bulbs. Once IGF-I or biologically active variant is dispensed into the nasal cavity, this agent can undergo transport through the nasal mucosa and into the tissues of the CNS.

Application of a pharmaceutical composition comprising a therapeutically effective amount or dose of IGF-I, or biologically active variant thereof, to a tissue innervated by the olfactory nerve can deliver this neuroprotective agent to damaged neurons or cells of the CNS, more particularly the region within the CNS that is affected by ischemia associated with an ischemic event. Olfactory neurons innervate this tissue and can provide a direct connection to the CNS, brain, and/or spinal cord due, it is believed, to their role in olfaction.

Delivery through the olfactory neural pathway can employ lymphatics that travel with the olfactory nerve to various brain areas and from there into dural lymphatics associated with portions of the CNS, such as the spinal cord. Transport along the olfactory nerve can also deliver this neuroprotective agent to an olfactory bulb. A perivascular pathway and/or a hemangiolymphatic pathway, such as lymphatic channels running within the adventitia of cerebral blood vessels, can provide an additional mechanism for transport of IGF-I or variant thereof to the brain and spinal cord from tissue innervated by the olfactory nerve. See International Publication No. WO 00/33813, herein incorporated by reference.

The pharmarceutical composition comprising IGF-I or biologically active variant thereof can be administered to the olfactory nerve, for example, through the olfactory epithelium. Such administration can employ extracellular or intracellular (e.g., transneuronal) anterograde and retrograde transport of the neurotrophic agent entering through the olfactory nerves to the brain and its meninges. Once this neuroprotective agent is dispensed into or onto tissue innervated by the olfactory nerve, the IGF-I or variant thereof may transport through the tissue and travel along olfactory neurons into areas of the CNS, more particularly the region of the brain affected by the ischemic event.

Delivery through the olfactory neural pathway can employ movement of IGF-I or variant thereof into or across mucosa or epithelium into the olfactory nerve or into a lymphatic, a blood vessel perivascular space, a blood vessel adventitia, or a blood vessel lymphatic that travels with the olfactory nerve to the brain and from there into meningial lymphatics associated with portions of the CNS such as the spinal cord. Blood vessel lymphatics include lymphatic channels that are around the blood vessels on the outside of the blood vessels. This also is referred to as the hemangiolymphatic system. Introduction of IGF-I or variant thereof into the blood vessel lymphatics does not necessarily introduce this neuroprotective agent into the blood.

In another embodiment of the invention, intranasal administration comprises administering IGF-I or biologically active variant thereof to tissue innervated by the trigeminal nerve and inside the nasal cavity. Within the nasal cavity, the trigeminal nerve innervates mainly the inferior two-thirds of the nasal mucosa. The trigeminal nerve has three major branches, the ophthalmic nerve, the maxillary nerve, and the mandibular nerve. The method of the invention can administer this neuroprotective agent to tissue within the nasal cavity innervated by one or more of these branches. See WO 00/33813, herein incorporated by reference.

The ophthalmic nerve has three branches known as the nasociliary nerve, the frontal nerve, and the lacrimal nerve. The anterior ethmoidal nerve, a branch of the nasociliary nerve, innervates, among other tissues, the ethmoidal sinus and regions of the inferior two-thirds of the nasal mucosa, including the anterior portion of the nasal septum and the lateral wall of the nasal cavity. Preferably, the method of the invention can administer IGF-I or biologically active variant thereof to tissue innervated by the anterior ethmoidal nerve.

The maxillary nerve has several branches that innervate the nasal cavity and sinuses, including the nasopalatine nerve, the greater palatine nerve, the posterior superior alveolar nerves, the middle superior alveolar nerve, and the interior superior alveolar nerve. The maxillary sinus is innervated by the posterior, middle, and anterior superior alveolar nerves. The mucous membrane of the nasal septum is supplied chiefly by the nasopalatine nerve, and the lateral wall of the nasal cavity is supplied by the greater palatine nerve. Preferably, the method of the invention can administer IGF-I or biologically active variant thereof to tissue innervated by the nasopalatine nerve and/or greater palatine nerve.

Application of the pharmaceutical composition comprising IGF-I, or biologically active variant thereof, to a tissue innervated by the trigeminal nerve can deliver this neuroprotective agent to damaged neurons or cells of the CNS, more particularly the region within the CNS that is affected by the ischemic event. Trigeminal neurons innervate the nasal cavity and can provide a direct connection to the CNS, brain, and/or spinal cord due, it is believed, to their role in the common chemical sense including mechanical sensation, thermal sensation and nociception (for example detection of hot spices and of noxious chemicals).

Delivery through the trigeminal neural pathway can employ lymphatics that travel with the trigeminal nerve to the pons and other brain areas and from there into dural lymphatics associated with portions of the CNS, such as the spinal cord. A perivascular pathway and/or a hemangiolymphatic pathway, such as lymphatic channels running within the adventitia of cerebral blood vessels, can provide an additional mechanism for transport of the neuroprotective agent to the spinal cord from tissue innervated by the trigeminal nerve.

The trigeminal nerve includes large diameter axons, which mediate mechanical sensation, e.g., touch, and small diameter axons, which mediate pain and thermal sensation, both of whose cell bodies are located in the semilunar (or trigeminal) ganglion or the mesencephalic trigeminal nucleus in the midbrain. Certain portions of the trigeminal nerve extend into the nasal cavity. Individual fibers of the trigeminal nerve collect into a large bundle, travel underneath the brain and enter the ventral aspect of the pons. The pharmaceutical composition comprising IGF-I or biologically active variant thereof can be administered to the trigeminal nerve, for example, through the nasal cavity's mucosa and/or epithelium. Such administration can employ extracellular or intracellular (e.g., transneuronal) anterograde and retrograde transport of this neuroprotective agent entering through the trigeminal nerve to the CNS tissues. Once dispensed into or onto intranasal tissue innervated by the trigeminal nerve, this neuroprotective agent may be transported through the tissue and travel along trigeminal neurons into areas of the CNS.

Delivery through the trigeminal neural pathway can employ movement of IGF-I or variant thereof across nasal mucosa or epithelium into the trigeminal nerve or into a lymphatic, a blood vessel perivascular space, a blood vessel adventitia, or a blood vessel lymphatic that travels with the trigeminal nerve to the pons and from there into meningial lymphatics associated with portions of the CNS such as the spinal cord.

Intranasal administration of IGF-I or biologically active variant thereof in accordance with the methods of the invention can more effectively deliver this therapeutic agent to the CNS, brain, and/or spinal cord, can decrease the amount of this agent administered outside the CNS, brain, and/or spinal cord, and, can preferably decrease the potential undesirable systemic effects of this agent. With more effective or efficient delivery to the CNS, brain, and/or spinal cord, the total dose of this agent that needs to be administered to provide a protective or therapeutic effect against ischemic damage associated with an ischemic event is decreased.

The term “IGF-I” as used herein refers to insulin-like growth factor I (IGF-I), a single-chain peptide having 70 amino acids and a molecular weight of about 7,600 daltons. Insulin-like growth factor I stimulates mitosis and growth processes associated with cell development.

In one embodiment of the invention, increasing the amount of IGF-I to a therapeutically effective level is achieved via administration of a pharmaceutical composition comprising a therapeutically effective dose. The IGF-I to be administered can be from any animal species including, but not limited to, rodent, avian, canine, bovine, porcine, equine, and, preferably, human. Preferably the IGF-I is from a mammalian species, and more preferably is from a mammal of the same species as the mammal undergoing treatment.

Biologically active variants of IGF-I are also encompassed by the method of the present invention. Such variants should retain IGF-I activities, particularly the ability to bind to IGF-I receptor sites. IGF-I activity may be measured using standard IGF-I bioassays. Representative assays include known radioreceptor assays using placental membranes (see, e.g., U.S. Pat. No. 5,324,639; Hall et al. (1974) J. Clin. Endocrinol. and Metab. 39:973-976; and Marshall et al. (1974) J. Clin. Endocrinol. and Metab. 39:283-292), a bioassay that measures the ability of the molecule to enhance incorporation of tritiated thymidine, in a dose-dependent manner, into the DNA of BALB/c 3T3 fibroblasts (see, e.g., Tamura et al. (1989) J. Biol. Chem. 262:5616-5621), and the like; herein incorporated by reference. Preferably, the variant has at least the same activity as the native molecule.

Suitable biologically active variants can be IGF-I fragments, analogues, and derivatives. By “IGF-I fragment” is intended a protein consisting of only a part of the intact IGF-I sequence and structure, and can be a C-terminal deletion or N-terminal deletion of IGF-I. By “analogue” is intended an analogue of either IGF-I or an IGF-I fragment that includes a native IGF-I sequence and structure having one or more amino acid substitutions, insertions, or deletions. Peptides having one or more peptoids (peptide mimics) are also encompassed by the term analogue (see e.g., International Publication No. WO 91/04282). By “derivative” is intended any suitable modification of IGF-I, IGF-I fragments, or their respective analogues, such as glycosylation, phosphorylation, or other addition of foreign moieties, so long as the IGF-I activity is retained. Methods for making IGF-I fragments, analogues, and derivatives are available in the art. See generally U.S. Pat. Nos. 4,738,921, 5,158,875, and 5,077,276; International Publication Nos. WO 85/00831, WO 92/04363, WO 87/01038, and WO 89/05822; and European Patent Nos. EP 135094, EP 123228, and EP 128733; herein incorporated by reference.

Preferably, naturally or non-naturally occurring IGF-I protein variants have amino acid sequences that are at least 70%, preferably 80%, more preferably, 85%, 90%, or 95% identical to the amino acid sequence of the reference IGF-I molecule, for example, the native human IGF-I, or to a shorter portion of the reference IGF-I molecule. More preferably, the molecules are 98% or 99% identical. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ by as few as 1 to 10 amino acid residues, such as 6-10, as few as 5, as few as 4, 3, 2, or even 1 amino aid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm noted above).

For example, conservative amino acid substitutions may be made at one or more predicted, preferably nonessential amino acid residues. A “nonessential” amino acid residue is a residue that can be altered from the wild-type or native sequence of IGF-I, such as human IGF-I, without altering its biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Such substitutions would not be made for conserved amino acid residues, or for amino acid residues residing within a conserved motif.

The art provides substantial guidance regarding the preparation and use of such IGF-I variants, as discussed further below. A fragment of IGF-I will generally include at least about 10 contiguous amino acid residues of the full-length molecule, preferably about 15-25 contiguous amino acid residues of the full-length molecule, and most preferably about 20-50 or more contiguous amino acid residues of full-length IGF-I.

Several IGF-I analogues and fragments are known in the art and include those described in, for example, Proc. Natl. Acad. Sci. USA 83(1986):4904-4907; Biochem. Biophys. Res. Commun. 149(1987):398-404; J. Biol. Chem. 263(1988):6233-6239; Biochem. Biophys. Res. Commun. 165(1989):766-771; Forsbert et al. (1990) Biochem. J. 271:357-363; U.S. Pat. Nos. 4,876,242 and 5,077,276; and International Publication Nos. WO 87/01038 and WO 89/05822. Representative analogues include one with a deletion of Glu-3 of the mature molecule, analogues with up to 5 amino acids truncated from the N-terminus, an analogue with a truncation of the first 3 N-terminal amino acids (referred to as des(1-3)-IGF-I, des-IGF-I, tIGF-I, or brain IGF), and an analogue including the first 17 amino acids of the B chain of human insulin in place of the first 16 amino acids of human IGF-I.

The IGF-I used in the present invention can be in its substantially purified, native, recombinantly produced, or chemically synthesized forms. For example, the IGF-I can be isolated directly from blood, such as from serum or plasma, by known methods. See, for example, Phillips (1980) New Eng. J. Med. 302:371-380; Svoboda et al. (1980) Biochemistry 19:790-797; Cornell and Boughdady (1982) Prep. Biochem. 12:57; Cornell and Boughdady (1984) Prep. Biochem. 14:123; European Patent No. EP 123,228; and U.S. Pat. No. 4,769,361. IGF-I may also be recombinantly produced. For example, the complete amino acid sequence of the human IGF-I protein is known, and DNA encoding human IGF-I has been cloned and expressed in E. coli and yeast. See, for example, U.S. Pat. Nos. 5,324,639, 5,324,660, and 5,650,496, and International Publication No. WO 96/40776, where recombinant production of human IGF-I in the yeast strain Pichia pastoris and purification of the recombinantly produced protein are described. Alternatively, IGF-I can be synthesized chemically, by any of several techniques that are known to those skilled in the peptide art. See, for example, Li et al. (1983) Proc. Natl. Acad. Sci. USA 80:2216-2220, Stewart and Young (1984) Solid Phase Peptide Synthesis (Pierce Chemical Company, Rockford, Ill.), and Barany and Merrifield (1980) The Peptides: Analysis, Synthesis, Biology, ed. Gross and Meienhofer, Vol. 2 (Academic Press, New York, 1980), pp. 3-254, for solid phase peptide synthesis techniques; and Bodansky (1984) Principles of Peptide Synthesis (Springer-Verlag, Berlin); and Gross and Meienhofer, eds. (1980) The Peptides: Analysis, Synthesis, Biology, Vol. 1 (Academic Press, New York), for classical solution synthesis. IGF-I can also be chemically prepared by the method of simultaneous multiple peptide synthesis. See, for example, Houghten (1985) Proc. Natl. Acad. Sci. USA 82:5131-5135; and U.S. Pat. No. 4,631,211. These references are herein incorporated by reference. Furthermore, methods to prepare a highly concentrated, low salt-containing, biologically active form of IGF-I or variant thereof are provided in International Publication No. WO 99/24062, entitled “Novel IGF-I Composition and Its Use,” herein incorporated by reference.

Thus, the IGF-I administered may be derived from any method known in the art. In one embodiment, the IGF-I administered is derived from a viscous syrup as described in WO 99/24062. Aliquots of this highly concentrated IGF-I syrup may be reconstituted into an injectable or infusible form such as a solution, suspension, or emulsion. It may also be in the form of a lyophilized powder, which can be converted into a solution, suspension, or emulsion before administration.

When administering IGF-I, appropriate serum glucose monitoring should be done to prevent hypoglycemia. The half-times of elimination, volumes of distribution, daily production rates, and serum concentrations are well established pharmacokinetic parameters for IGF-I in normal humans. See, for example, Guler et al. (1989) Acta Endocrinological (Copen) 121:753-58); Zapfet al. (1981) J. Clin. Invest. 68:1321-30.

Increases in the amount of IGF-I or biologically active variant thereof in the CNS, brain, and/or spinal cord to a therapeutically effective level may be obtained via administration of a pharmaceutical composition including a therapeutically effective dose of this agent. By “therapeutically effective dose” is intended a dose of IGF-I or biologically active variant thereof that achieves the desired goal of increasing the amount of this agent in a relevant portion of the CNS, brain, and/or spinal cord to a therapeutically effective level enabling a desired biological activity of IGF-I or biologically active variant thereof. Desired biological activities beneficial to treatment of ischemic damage include, for example, an increase in protein phosphorylation, particularly of the IGF-I receptor, in response to IGF-I (see, for example, International Publication No. WO 00/33813), or other activities such as increasing choline acetyltransferase activity and enhancing neuronal survival (see, for example, U.S. Pat. No. 5,652,214).

When delivered to the region of the CNS that has suffered the initial ischemic damage associated with an ischemic event, a therapeutically effective amount or dose of IGF-I or biologically active variant thereof results in a reduction in the amount of further ischemic damage in the region. This reduction in ischemic damage can be measured as a decrease in infarct size and edema. Further, because CNS tissue damage is decreased, a mammal undergoing treatment in accordance with the methods of the present invention exhibits a reduction in neural deficits, and hence improved recovery of motor, sensory, vestibulomoter, and/or somatosensory function. Similarly, when a therapeutically effective amount of IGF-I is administered intranasally to a mammal at risk of experiencing an ischemic event prior to the event, ischemic damage can be prevented if the ischemic event occurs.

Methods to quantify the extent of ischemic damage and to determine if an ischemic event has been treated, particularly with regard to reduction or prevention of ischemic damage including infarct size, edema, and development of neural deficits, are well known to those skilled in the art. Such methods include, but are not limited to, histological methods, molecular marker assays, and functional/behavior analysis. For example, after ischemic injury, there is a significant increase in the density of omega 3 (peripheral-type benzodiazepine) binding sites (Benazodes et al. (1990) Brain Res. 522:275-289). Methods to detect omega 3 sites are known and can be used to determine the extent of ischemic damage. See for example, Gotti et al. (1990) Brain Res. 522:290-307 and references cited therein. Alternatively, Growth Associated Protein-43 (GAP-43) can be used as a marker for new axonal growth following an ischemic event. See, for example, Stroemer et al. (1995) Stroke 26:2135-2144, and Vaudano et al. (1995) J. Neurosci 15:3594-3611. The therapeutic effect may also be measured by improved motor skills, cognitive function, sensory perception, speech and/or a decrease in the propensity to seizure in the mammal undergoing treatment. Such functional/behavior tests used to assess sensorimotor and reflex function are described in, for example, Bederson et al. (1986) Stroke 17:472-476, DeRyck et al. (1992) Brain Res. 573:44-60, Markgraf et al. (1992) Brain Res. 575:238-246, Alexis et al. (1995) Stroke 26:2338-2346. Enhancement of neuronal survival may also be measured using the Scandinavian Stroke Scale (SSS) or the Barthel Index.

The present invention is not held to any particular mechanism of neuroprotection for the IGF-I or variant thereof. It is, however, believed that the neuroprotective properties of IGF-I result from the suppression of events that occur in the latent phase of programmed cell death (D'Mello et al. (1993) Proc. Natl. Acad. Sci. 90:10989-10993 and Samejima et al. (1998) J. Cell. Biol. 143:225-239). Evidence suggests IGF-I suppresses apoptosis through mechanisms involving the phosphatidylinositol 3′-kinase and mitogen-activated protein kinase pathways. See, for example, Parrivas (1997) J. Biol. Chem. 272:154-161. The inhibition of cell death by IGF-I is broadly based, with evidence that IGF-I can block otherwise neurotrophin insensitive apoptosis (Fernandezsanchez et al. (1996) FEBS Lett. 398:106-112).

By “therapeutically effective amount” or “dose” is meant the concentration of IGF-I or biologically active variant thereof that is sufficient to elicit the desired therapeutic effect with respect to reducing or preventing ischemic damage associated with an ischemic event described elsewhere herein. The therapeutic effective amount will depend on many factors including, for example, the severity and pattern of ischemic damage, the responsiveness of the subject undergoing treatment, the weight of the subject, and the amount of time that lapsed between the ischemic event and the administration of the IGF-I or variant thereof. Methods to determine efficacy and dosage are known to those skilled in the art.

For purposes of reducing or preventing ischemic damage associated with an ischemic event in a mammal in need thereof, the therapeutically effective amount or dose of IGF-I or biologically active variant thereof is about 0.10 mg to about 3.0 mg per kg body weight, preferably about 0.15 mg to about 2.8 mg per kg body weight, more preferably about 0.20 mg to about 2.6 mg per kg, even more preferably about 0.25 mg to about 2.4 mg per kg, still more preferably about 0.50 mg to about 2.0 mg per kg, yet more preferably about 0.80 mg to about 2.0 mg per kg, still more preferably about 1.0 mg to about 2.0 mg per kg body weight, even more preferably about 1.0 mg to about 1.5 mg per kg body weight. In some regimens, therapeutically effective doses for administration of IGF-I or variant thereof to a human include about 0.10, 0.20, 0.25, 0.30, 0.35, 0.40, 0.45, 0.50, 0.55, 0.60, 0.65, 0.70, 0.75, 0.80, 0.85, 0.90, 0.95, 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mg per kg body weight. These doses depend on factors including the efficiency with which IGF-I or biologically active variant thereof is transported from the nasal cavity to the CNS. A larger total dose can be delivered by multiple administrations of the agent.

In addition to the therapeutically effective dose of IGF-I or biologically active variant thereof, the composition can include, for example, any pharmaceutically acceptable additive, carrier, and/or adjuvant that can promote the transfer of this neuroprotective agent within or through the mucosa or epithelium of the nasal cavity, or along or through a neural pathway. Alternatively, the composition can comprise IGF-I or biologically active variant thereof combined with substances that assist in transporting IGF-I or biologically active variant thereof to sites of nerve cell damage. The composition can further comprise additional neurotrophic agents, such as nerve growth factor (NGF), fibroblast growth factor, and the like, so long as the therapeutic efficacy of IGF-I or biologically active variant thereof against stroke is not lessened.

By “pharmaceutically acceptable carrier” is intended a carrier that is conventionally used in the art to facilitate the storage, administration, and/or the healing effect of IGF-I or biologically active variant thereof. A carrier may also reduce any undesirable side effects of this neurotrophic agent. A suitable carrier should be stable, i.e., incapable of reacting with other ingredients in the formulation. It should not produce significant local or systemic adverse effect in recipients at the dosages and concentrations employed for treatment. Such carriers are generally known in the art.

Suitable carriers for this invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like.

Other acceptable components in the composition include, but are not limited to, isotonicity-modifying agents such as water, saline, and buffers including phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein-based compositions, particularly pharmaceutical compositions, is well known in the art. See, Wang et al. (1980) J. Parent. Drug Assn. 34(6):452-462; Wang et al. (1988) J. Parent. Sci. and Tech. 42:S4-S26 (Supplement); Lachman et al. (1968) Drug and Cosmetic Industry 102(1):36-38, 40, and 146-148; Akers (1988) J. Parent. Sci. and Tech. 36(5):222-228; and Methods in Enzymology, Vol. XXV, ed. Colowick and Kaplan, “Reduction of Disulfide Bonds in Proteins with Dithiothreitol,” by Konigsberg, pages 185-188.

Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) supra at page 455. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

Suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. See Akers (1988) supra at pages 225-226. Suitable antioxidants include sodium bisulfite, sodium sulfite, sodium metabisulfite, sodium thiosulfate, sodium formaldehyde sulfoxylate, and ascorbic acid. See Akers (1988) supra at page 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See, e.g., Wang (1980) supra at pages 457-458 and 460-461, and Akers (1988) supra at pages 224-227.

The composition can include one or more preservatives such as phenol, cresol, paraaminobenzoic acid, BDSA, sorbitrate, chlorhexidine, benzalkonium chloride, or the like. Suitable stabilizers include carbohydrates such as trehalose or glycerol. The composition can include a stabilizer such as one or more of microcrystalline cellulose, magnesium stearate, mannitol, or sucrose to stabilize, for example, the physical form of the composition; and one or more of glycine, arginine, hydrolyzed collagen, or protease inhibitors to stabilize, for example, the chemical structure of the composition. Suitable suspending agents include carboxymethyl cellulose, hydroxypropyl methylcellulose, hyaluronic acid, alginate, chondroitin sulfate, dextran, maltodextrin, dextran sulfate, or the like. The composition can include an emulsifier such as polysorbate 20, polysorbate 80, pluronic, triolein, soybean oil, lecithins, squalene and squalanes, sorbitan treioleate, or the like. The composition can include an antimicrobial such as phenylethyl alcohol, phenol, cresol, benzalkonium chloride, phenoxyethanol, chlorhexidine, thimerosol, or the like. Suitable thickeners include natural polysaccharides such as mannans, arabinans, alginate, hyaluronic acid, dextrose, or the like; and synthetic ones like the PEG hydrogels of low molecular weight; and aforementioned suspending agents.

The composition can include an adjuvant such as cetyl trimethyl ammonium bromide, BDSA, cholate, deoxycholate, polysorbate 20 and 80, fusidic acid, or the like. Suitable sugars include glycerol, threose, glucose, galactose, mannitol, and sorbitol.

Preferred compositions include one or more of a solubility enhancing additive, preferably a cyclodextrin; a hydrophilic additive, preferably a monosaccharide or oligosaccharide; an absorption promoting additive, preferably a cholate, a deoxycholate, a fusidic acid, or a chitosan; a cationic surfactant, preferably a cetyl trimethyl ammonium bromide; a viscosity enhancing additive, preferably to promote residence time of the composition at the site of administration, preferably a carboxymethyl cellulose, a maltodextrin, an alginic acid, a hyaluronic acid, or a chondroitin sulfate; or a sustained release matrix, preferably a polyanhydride, a polyorthoester, a hydrogel, a particulate slow release depo system, preferably a polylactide co-glycolides (PLG), a depo foam, a starch microsphere, or a cellulose derived buccal system; a lipid-based carrier, preferably an emulsion, a liposome, a niosome, or a micelle. The composition can include a bilayer destabilizing additive, preferably a phosphatidyl ethanolamine; a fusogenic additive, preferably a cholesterol hemisuccinate.

These lists of carriers and additives are by no means complete, and a worker skilled in the art can choose excipients from the GRAS (generally regarded as safe) list of chemicals allowed in the pharmaceutical preparations and those that are currently allowed in topical and parenteral formulations.

For the purposes of this invention, the pharmaceutical composition comprising IGF-I or biologically active variant thereof can be formulated in a unit dosage and in a form such as a solution, suspension, or emulsion. The pharmaceutical composition to be administered to the nasal cavity may be in the form of a powder, a granule, a solution, a cream, a spray (e.g., an aerosol), a gel, an ointment, an infusion, an injection, a drop, or a sustained-release composition, such as a polymer disk. Other forms of compositions for administration include a suspension of a particulate, such as an emulsion, a liposome, an insert that releases the neurotrophic agent slowly, and the like. The powder or granular forms of the pharmaceutical composition may be combined with a solution and with a diluting, dispersing, or surface active neurotrophic agent. Additional preferred compositions for administration include a bioadhesive to retain the IGF-I or biologically active variant thereof at the site of administration; a spray, paint, or swab applied to the mucosa or epithelium; or the like. The composition can also be in the form of lyophilized powder, which can be converted into solution, suspension, or emulsion before administration. The pharmaceutical composition comprising IGF-I or variant thereof is preferably sterilized by membrane filtration and is stored in unit-dose or multi-dose containers such as sealed vials or ampoules.

The method for formulating a pharmaceutical composition is generally known in the art. A thorough discussion of formulation and selection of pharmaceutically acceptable carriers, stabilizers, and isomolytes can be found in Remington 's Pharmaceutical Sciences (18^{th} ed.; Mack Publishing Company, Eaton, Pa., 1990), herein incorporated by reference.

The IGF-I or biologically active variant thereof can also be formulated in a sustained-release form to prolong the presence of this pharmaceutically active component in the treated mammal, generally for longer than one day. Many methods of preparation of a sustained-release formulation are known in the art and are disclosed in Remington's Pharmaceutical Sciences (18^{th} ed.; Mack Publishing Company, Eaton, Pa., 1990), herein incorporated by reference.

Generally, IGF-I or biologically active variant thereof can be entrapped in semipermeable matrices of solid hydrophobic polymers. The matrices can be shaped into films or microcapsules. Examples of such matrices include, but are not limited to, polyesters, copolymers of L-glutamic acid and gamma ethyl-L-glutamate (Sidman et al. (1983) Biopolymers 22: 547-556), polylactides (U.S. Pat. No. 3,773,919 and EP 58,481), polylactate polyglycolate (PLGA) such as polylactide-co-glycolide (see, for example, U.S. Pat. Nos. 4,767,628 and 5,654,008), hydrogels (see, for example, Langer et al. (1981) J. Biomed. Mater. Res. 15: 167-277; Langer (1982) Chem. Tech. 12: 98-105), non-degradable ethylene-vinyl acetate (e.g., ethylene vinyl acetate disks and poly(ethylene-co-vinyl acetate)), degradable lactic acid-glycolic acid copolyers such as the Lupron Depot™poly-D-(−)-3-hydroxybutyric acid (EP 133,988), hyaluronic acid gels (see, for example, U.S. Pat. No. 4,636,524), alginic acid suspensions, and the like.

Suitable microcapsules can also include hydroxymethylcellulose or gelatin-microcapsules and polymethyl methacrylate microcapsules prepared by coacervation techniques or by interfacial polymerization. See International Publication No. 99/24061, entitled “Method for Producing Sustained-release Formulations,” wherein proteins are encapsulated in PLGA microspheres, herein incorporated by reference. In addition, microemulsions or colloidal drug delivery systems such as liposomes and albumin microspheres, may also be used. See Remington's Pharmaceutical Sciences (18^{th} ed.; Mack Publishing Company Co., Eaton, Pa., 1990). Other sustained-release compositions employ a bioadhesive to retain the pharmacologically active agent at the site of administration.

Among the optional substances that may be combined with IGF-I or variant thereof in the pharmaceutical composition are lipophilic substances that can enhance absorption of this neuroprotective agent through the mucosa or epithelium of the nasal cavity to damaged cells in the CNS. The neuroprotective agent may be mixed with a lipophilic adjuvant alone or in combination with a carrier, or may be combined with one or several types of micelle or liposome substances. Among the preferred lipophilic substances are cationic liposomes including one or more of phosphatidyl choline, lipofectin, DOTAP, or the like. These liposomes may include other lipophilic substances such as gangliosides and phosphatidylserine (PS). Also preferred are micellar additives such as GM-I gangliosides and phosphatidylserine (PS), which may be combined with the neurotrophic agent either alone or in combination. GM-1 ganglioside can be included at 1-10 mole percent in any liposomal compositions or in higher amounts in micellar structures. Protein agents can be either encapsulated in particulate structures or incorporated as part of the hydrophobic portion of the structure depending on the hydrophobicity of the protein agent. One preferred liposomal formulation employs Depofoam. The neuroprotective agent can be encapsulated in multivesicular liposomes, as disclosed in International Publication No. WO 99/12522, entitled “High and Low Load Formulations of IGF-I in Multivesicular Liposomes,” herein incorporated by reference.

The pharmaceutical composition may additionally include a solubilizing compound to enhance stability of IGF-I or biologically active variant thereof. For IGF-I, a preferred solubilizing agent includes a guanidinium group that is capable of enhancing its solubility. Examples of such solubilizing compounds include the amino acid arginine, as well as amino acid analogs of arginine that retain the ability to enhance solubility of IGF-I or biologically active variant thereof at pH 5.5 or greater. Such analogs include, without limitation, dipeptides and tripeptides that contain arginine. By “enhancing the solubility” is intended increasing the amount of IGF-I or biologically active variant thereof that can be dissolved in solution at pH 5.5 or greater in the presence of a guanidinium-containing compound compared to the amount of this protein that can be dissolved at pH 5.5 or greater in a solution with the same components but lacking the guanidinium-containing compound. The ability of a guanidinium-containing compound to enhance the solubility of IGF-I or biologically active variant thereof can be determined using methods well known in the art. In general, the concentration of the solubilizing compound present in the composition will be from about 10 mM to about 1 M, and, for example, in the case of the compound arginine, in a concentration range of about 20 mM to about 200 mM, as disclosed in International Publication No. WO 99/24063, entitled “Compositions Providing for Increased IGF-I Solubility,” herein incorporated by reference.

In one embodiment, the composition includes the combination of an effective amount of IGF-I with poly(ethylene-co-vinyl acetate) to provide for controlled release of this neuroprotective agent.

A composition formulated for intranasal delivery may optionally comprise an odorant. An odorant agent is combined with the neuroprotective agent to provide an odorliferous sensation, and/or to encourage inhalation of the intranasal preparation to enhance delivery of IGF-I or biologically active variant thereof to the olfactory neuroepithelium. The odoriferous sensation provided by the odorant agent may be pleasant, obnoxious, or otherwise malodorous. The odorant receptor neurons are localized to the olfactory epithelium, which, in humans, occupies only a few square centimeters in the upper part of the nasal cavity. The cilia of the olfactory neuronal dendrites which contain the receptors are fairly long (about 30-200 um). A 10-30 um layer of mucus envelops the cilia that the odorant agent must penetrate to reach the receptors. See Snyder et al. (1988) J. Biol. Chem. 263:13972-13974. Use of a lipophilic odorant agent having moderate to high affinity for odorant binding protein (OBP) is preferred. OBP has an affinity for small lipohilic molecules found in nasal secretions and may act as a carrier to enhance the transport of a lipohilic odorant substance and active neuroprotective agent to the olfactory receptor neurons. It is also preferred that an odorant agent is capable of associating with lipophilic additives such as liposomes and micelles within the preparation to further enhance delivery of the neuroprotective agent by means of OBP to the olfactory neuroepithelium. OBP may also bind directly to lipophilic agents to enhance transport of the neuroprotective agent to olfactory neural receptors.

Suitable odorants having a high affinity for OBP include terpanoids such as cetralva and citronellol, aldehydes such as amyl clnnamaldehyde and hexyl cinnamaldehyde, esters such as octyl isovalerate, jasmines such as C1S-jasmine and jasmal, and musk 89. Other suitable odorant agents include those which may be capable of stimulating odorant-sensitive enzymes such as aderrylate cyslase and guanylate cyclase, or which may be capable of modifying ion channels within the olfactory system to enhance absorption of the neuroprotective agent.

The pharmaceutical composition having a unit dose of IGF-I or biologically active variant thereof can be, for example, in the form of solution, suspension, emulsion, or a sustained-release formulation. Preferably, the total volume of one dose of the pharmaceutical composition ranges from about 10 μl to about 0.2 ml, preferably from about 50 μl to about 200 μl. It is apparent that the suitable volume can vary with factors such as the size of the nasal cavity to which IGF-I or biologically active variant thereof is administered and the solubility of the components in the composition.

It is recognized that the total amount of IGF-I or variant thereof administered as a unit dose to a particular tissue will depend upon the type of pharmaceutical composition being administered, that is whether the composition is in the form of, for example, a solution, a suspension, an emulsion, or a sustained-release formulation. For example, where the pharmaceutical composition comprising a therapeutically effective amount of this neuroprotective agent is a sustained-release formulation, the neuroprotective agent is administered at a higher concentration.

It should be apparent to a person skilled in the art that variations may be acceptable with respect to the therapeutically effective dose and frequency of the administration of the neuroprotective agent in this embodiment of the invention. The amount of the neuroprotective agent administered will be inversely correlated with the frequency of administration. Hence, an increase in the concentration of IGF-I or variant thereof in a single administered dose, or an increase in the mean residence time in the case of a sustained release form of the neuroprotective agent, generally will be coupled with a decrease in the frequency of administration.

It is recognized that a single dosage of the neuroprotective agent may be administered over the course of several minutes, hours, days, or weeks. A single dose of the neuroprotective agent may be sufficient. Alternatively, repeated doses may be given to a patient over the course of several hours, days or weeks. In addition, if desired, a combination of neuroprotective agents may be administered as noted elsewhere herein.

Further, the therapeutically effective amount or dose of IGF-I or variant thereof and the frequency of administration will depend on whether it is administered for purposes of reducing ischemic damage in a subject that has experienced an ischemic event, or for purposes of preventing ischemic damage in a subject that is at risk of experiencing an ischemic event. Thus, one of skill in the art would recognize that higher doses would be administered to a mammal that has already suffered an ischemic event and the objective is to reduce ischemic damage. Similarly, a skilled artisan would recognize that lower doses would be administered to a mammal that is at risk of experiencing the ischemic event and the objective is to prevent ischemic damage if the ischemic event occurs.

Some minor degree of experimentation may be required to determine the most effective dose and frequency of dose administration, this being well within the capability of one skilled in the art once apprised of the present disclosure. The method of the present invention may be used with any mammal. Exemplary mammals include, but are not limited to rats, cats, dogs, horses, cows, sheep, pigs, and more preferably humans.

For purposes of reducing ischemic damage in a mammal that has experienced an ischemic event, intranasal administration of one or more therapeutically effective doses of IGF-I or variant thereof may occur within minutes, hours, days, or even weeks of the initial ischemic event. For example, the initial therapeutic dose may be administered within about 2 to 4 hours, within about 2 to 6 hours, within about 8 hours, within about 10 hours, about 15 hours, about 24 hours, within about 36 hours, 48 hours, 72 hours, or about 96 hours, and one or more additional doses may be administered for hours, days, or weeks following the initial dose. Where the mammal is at risk of experiencing an ischemic event, administration may occur within weeks, days, hours, or minutes prior to event occurring. Thus, for example, a mammal undergoing a cardiovascular surgical procedure can be administered one or more therapeutically effective doses of IGF-I or biologically active variant thereof prior to, during, or following the surgical procedure.

The present invention may be better understood with reference to the following examples. These examples are intended to be representative of specific embodiments of the invention, and are not intended as limiting the scope of the invention.

### Experimental

### EXAMPLE 1

### Intranasal Administration of IGF-I Protects Against Focal Cerebral Ischemic Damage in Rats with MCAO

The following study was undertaken to determine the dose-response relationship of intranasally administered IGF-I in an animal model of stroke.

### Materials and Methods

Animal Preparation

All procedures used in this and the following example were approved by the Animal Care and Use Committee at Regions Hospital and complied with the Principles of Laboratory Animal Care Guidelines (NIH publication #85-23, revised 1985). A total of 77 adult male Sprague-Dawley rats weighing 250-303 g, were anesthetized with 3% halothane for induction and 1.5% halothane in oxygen for maintenance. During surgical procedures, animal temperature was continuously monitored with a rectal probe and maintained at 37° C. with a heating pad.

Middle Cerebral Artery Occlusion

Focal brain ischemia was induced by the intraluminal suture MCAO method as previously described (Longa et al. (1989) Stroke 20:84-91; Chen et al. (1992) J. Cereb. Blood Flow Metab. 12:621-8). Briefly, the right common carotid artery (CCA), external carotid artery (ECA), and internal carotid artery (ICA) were exposed. A length (19±0.5 mm) of 4-0 nonofilament nylon suture, determined by the animal weight, with its tip rounded by heating near a flame, was advanced from the ECA into the lumen of ICA until it blocked the origin of the middle cerebral artery (MCA). Two hours after MCAO, animals were reanesthetized with halothane and reperfusion was performed by withdrawal of the suture until the tip cleared the lumen of the ICA.

Drug Preparation:

IGF-I for use in this and the following example was recombinantly produced in the yeast strain Pichia pastoris and purified essentially as described in U.S. Pat. Nos. 5,324,639, 5,324,660, and 5,650,496 and International Publication No. WO 96/40776, herein incorporated by reference. The rIGF-I was prepared at various concentrations in a 10 mM sodium succinate buffer at pH 6.0, with 140 mM sodium chloride. The vehicle was 10 mM sodium succinate buffer at pH 6.0, with 140 mM sodium chloride.

Protocol

A total of three studies were performed. In the pilot study, the investigator was not blinded. The rats were divided into two groups, 75 μg IGF-I treated (n=11) and vehicle control (n=10). During the 72 hour survival, neurologic deficit was only assessed by Longa's test (Longa et al. (1989) Stroke 20:84-91). The results showed that neurologic function was statistically significantly improved at 48 hours and 72 hours (p<0.04) after the onset of MCAO (data not shown). The following two studies were conducted with the investigator blinded. The first of these studies repeated the pilot study with a 75 μg IGF-I treated group (n=10), and the vehicle control-I group (n=12). The second study used IGF-I doses of 150 μg (n=10), 37.5 μg (n=12), and vehicle control-2 (n=12) to explore the dose-dependent relationship.

Intranasal Administration

Recombinant human (rh) IGF-I (rh-IGF-I) and vehicle were provided by Chiron Corporation (Emeryville, Calif.). The rats were randomly divided into different groups (study 1: 75 μg rh-IGF-I and the vehicle control-1; study 2: 150 μg rh-IGF-I, 37.5 μg rh-IGS-1, and the vehicle control-2). In order to blind the investigator, who assessed the neurologic deficit function and infarct volume, the IGF-I and vehicle solutions to be administered to the animals were coded. Thus, the investigator did not know which solution was IGF-I or vehicle until all the neurologic functions and infarct volumes had been statistically analyzed.

Within each treatment or control group, three doses of the respective treatment (37.5, 75, or 150 μg rh-IGF-I) or vehicle solutions were administered to each rat as follows. For each dose, a total of 50 μl solution were given in nose drops (5 μl per drop) over a 20 minute period, alternating drops every two minutes between the left and right nares. During the administration, the mouth and the opposite naris were held closed so the drops could be naturally inhaled. This method of administering allows for both pressure and gravity to deliver the agent (i.e., IGF-I or vehicle) into the upper one-third of the nasal cavity. Ten minutes after initiating MCAO, the halothane anesthetized rats were placed on their backs and administered a first dose of the respective treatment. The second and third doses were administered in the same manner 24 hours and 48 hours, respectively, after the onset of MCAO.

Behavioral Testing

Following recovery from anesthesia, behavioral neurologic deficits were assessed at 4, 24, 48, and 72 hours after the onset of MCAO. Four behavioral tests were used to systematically evaluate motor, sensory, and vestibulomotor deficits: Postural reflex and hemiparesis test with a 4-point scale (0-4) described by Bederson et al. (1986) Stroke 17:472-476: 0 no observable neurologic deficits, 1=left forepaw flexion, 2=decreased resistance to lateral push and forepaw flexion without circling, 3=same behavior as 2 with circling, 4=cannot walk spontaneously.

Forepaw placing test based on DeRyck et al.(1988) Stroke 20:1383-1390: Visual forepaw placing was tested first by lowering the rat toward a table top and by contacting the table edge with the dorsal or lateral aspect of the rat's forepaw. Non-visual forepaw placing was also tested by lowering the rat toward the table top and by contacting the table edge respectively when cephalic contact stimuli were eliminated by lifting up the rat's head 45°. For each test, limb placing scores were: 0=immediate and complete placing, 1=delayed and/or incomplete (>2 seconds), 2=no placing.

Modified beam balance test (Feeney et al. (1982) Science 217:855-857; Dixion et al. (1987) J. Neurosurg. 67:110-119): The rat was placed on a narrow beam (40×1.3×1.3 cm, 30 cm above the table top) for 60 seconds. At least five training scores were recorded before MCAO. The functional deficit scale was as follows: 1=steady posture with paws on top of the beam, 2=paws on side of beam or wavering, 3=one or two limb(s) slip off, 4=three limbs slip off, 5=attempts with paws on the beam, but falls, 6=drapes over the beam, then falls or falls with no attempt.

Adhesive tape test (Schallert et al. (1982) Pharmacol. Biochem. Behav. 16:445462; Herandez and Schaller (1988) Exp. Neurol. 102:318-324; Andersen et al. (1990) Physiol. Behav. 47:1045-1052): Somatosensory deficit was measured both pre-and postoperatively. All rats were familiarized with the testing environment. In the initial test, two small pieces of adhesive-backed paper dots (of equal size, 113.1 mm²) were used as bilateral tactile stimuli respectively occupying the ventral side of each forepaw. The rat was then returned to its cage. The latencies to contact and remove each stimulus from the paw were recorded on five trials per day. Individual trials were separated by at least 10 min. The average time of the five trials was used for each day's record. The animals were trained two days prior to surgery. Once the rats were able to remove the paper dots within 10 seconds(s), they were subjected to MCAO. The scale was evaluated as: 1=<10 s; 2=10-19 s; 3=20-29 s; 4=30-39 s; 5=40-49 s; 6=50-59 s, 7=260 s.

Histological Assessment

Rats were allowed to survive for 72 hours at which time the rats were euthanized with 5% halothane/oxygen. The rat brains were fixed by transcardial perfusion with normal saline, followed by neutral buffered 10% formalin. Each brain was carefully removed and immersed in 10% formalin solution for at least 48 hours, and then sectioned into seven equally spaced (2 mm) coronal sections from the frontal lobe to the occipital lobe. Brain sections were embedded in paraffin. A series of adjacent 5-micrometer-thick sections were cut from each section in the coronal plane and stained with hematoxolin and eosin. An image analysis system (AIS/C Imagine Research Inc., St. Catherines, Ontario, Canada) was used to determine the measured infarct volume (MV), right hemisphere volume (RV), and left hemisphere volume (LV). The infarct volume was calculated by numeric integration of data from individual slices. To compensate for brain swelling in the ischemic hemisphere, the corrected infarct volume in each rat was determined by a previously described formula (Swanson et al. (1990) J. Cereb. Blood Flow Metab. 10:290-293; Aspey et al (1998) Neuropathol. Appl. Neurobiol. 24:487-497) corrected infarct volume (%)=[LV−(RV−MV)]/LV×100. The morphologic changes of the infarct area, penumbra, and non-ischemic areas were observed by light microscopy.

Statistics

Separate analyses were performed for the two studies with study 1 comparing the 75 μg rh-IGF-I and control-1 groups and study 2 comparing the 150 μg rh-IGF-I, 37.5 μg rh-IGF-I and control-2 groups. Mortality rates were compared between the groups using Fisher's exact test. Baseline body weight and corrected infarct volume were compared between groups using t-tests (study-1) and analyses of variance (study-2). Neurologic behavioral tests and body weight changes were analyzed using repeated measure analysis of variance (ANOVA) models. Separate analysis of variance models (study 2) and Wilcoxon rank-sum tests (study 1) were performed for each of the time points to see at what time point the scores became significantly different (significance level of 0.02 used to take into account the multiple comparisons). Pearson correlation coefficients were calculated between the infarct volume and behavioral tests at the 72-hour point.

### Results

Mortality Rate

Combining the three studies (pilot+study 1+study 2), the overall mortality rates were 0% (0/10), 5% (1/21), 17% (2/12), and 24% (8/34) in the 150, 75, 37.5 μg rh-IGF-I and control groups, respectively. These rates were not found to be statistically significantly different (p=0.139). Collapsing the four groups into an IGF-I effective group (150 μg+75 μg) and an IGF-I ineffective group (37.5 μg+controls), the mortality rates were 3% and 22% respectively. These collapsed rates were found to be statistically significantly different (p=0.042). This suggests that IN administration of an effective dose of IGF-I reduces the mortality rate in rats with stroke.

Effect of IN IGF-I on the Corrected Infarct Volume

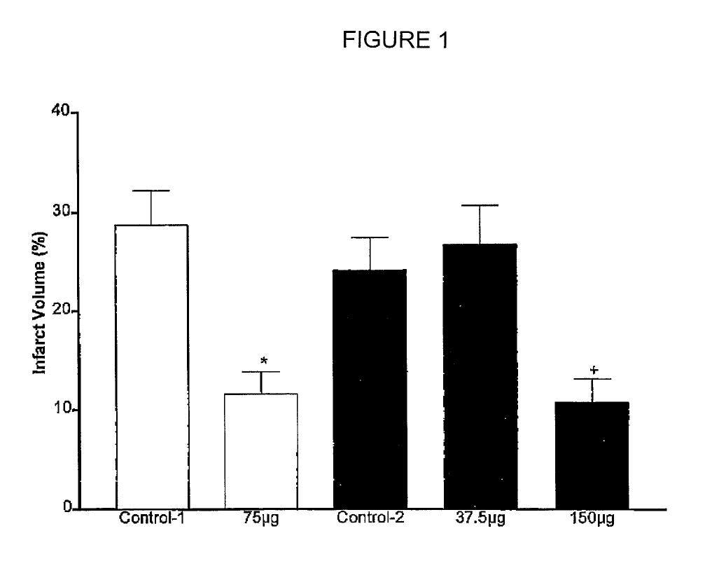

Both 150 μg and 75 μg doses of rh-IGF-I significantly reduced the infarct volume, while the 37.5 μg dose was ineffective. For study 1, the average infarct volumes were 11.5% and 28.8% in the 75 μg rh-IGF-I and control-I groups, respectively. For study 2, the average infarct volumes were 10.7%, 26.7%, and 22.5% in the 150 μg rh-IGF-I, 37.5 μg rh-IGF-I, and control-2 groups, respectively. The 75 μg rh-IGF-I group had significantly lower infarct volume than the control-1 (p=0.001). The 150 μg rh-IGF-I group had significantly lower infarct volume than both the 37.5 μg rh-IGF-I and control-2 groups (p=0.004). The 37.5 μg rh-IGF-I and control-2 groups were not significantly different. Variability in the dose-dependent relationship might have been due to the 75 μg rh-IGF-I and control-I groups not being performed at the same time as the 150 μg and 37.5 μg rh-IGF-I and control-2 groups (FIG. 1).

Effect of IN IGF-I on Motor-Sensory Function as Assessed by the Postural Reflex and Hemiparesis Test

There was a significant difference in the postural reflex and hemiparesis test scores between the 150 μg, 37.5 μg, and control-2 groups (p=0.001), with the 150 μg rhIGF-I group having lower scores on average than the 37.5 μg rh-IGF-I and control-2 groups. There was a significant change in scores over time (p=0.001), and the change over time was significantly different between the groups (p=0.001). The average scores in the 150 μg rh-IGF-I group were significantly lower than that in the 37.5 μg rh-IGF-I and control-2 groups at 24 hours (p=0.02), 48 hours (p=0.001), and 72 hours (p=0.001). There was no significant difference between the groups at 4 hours.

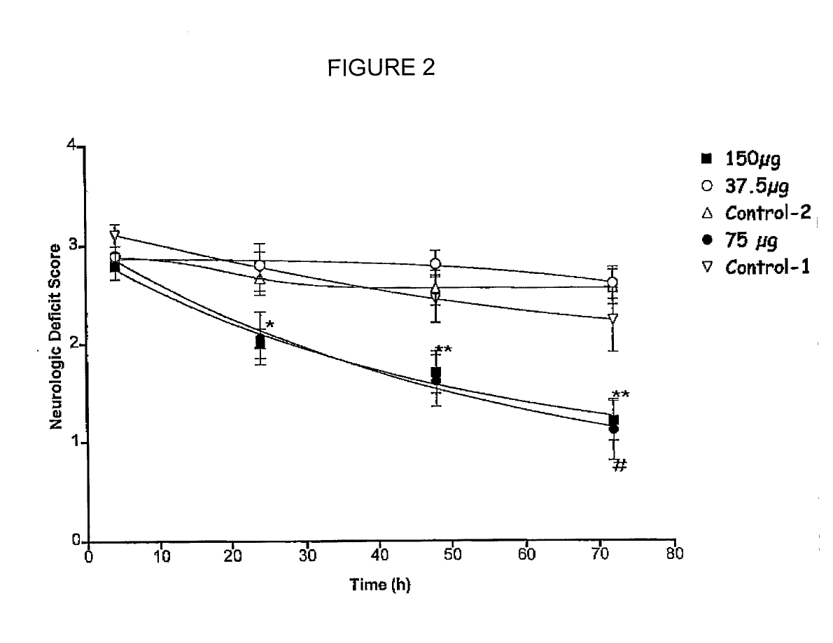

There was a significant difference in the postural reflex and hemiparesis test scores between the 75 μg rh-IGF-I and control-1 groups (p=0.020), with the 75 μg rhIGF-I group having lower scores than the control-1 group. There was a significant change in scores over time (p=0.001), and the change over time was not significantly different between the groups (p=0.073). The scores in the 75 μg rh-IGF-I group were borderline significantly lower than that in the control-1 group at 72 hours (p=0.027). There was no significant difference between the groups at 4 hours, 24 hours, and 48 hours (FIG. 2).

Effect of IN IGF-I on Sensorimotor Performance Assessed by the Placing Test

There, was a significant difference in the left forepaw placing test scores between the 150 μg, 37.5 μg, and control-2 groups (p=0.021), with the 150 μg rh-IGF-I group having lower scores on average than the 37.5 μg rh-IGF-I group. There was a significant change in scores over time (p=0.001), and the change over time was not significantly different between the groups (p=0.215). The average score in the 150 μg rh-IGF-I group was significantly lower than that in the 37.5 μg rh-IGF-I and control-2 groups at 72 hours (p=0.008). There was no significant difference between the groups at 24 hours and 48 hours.

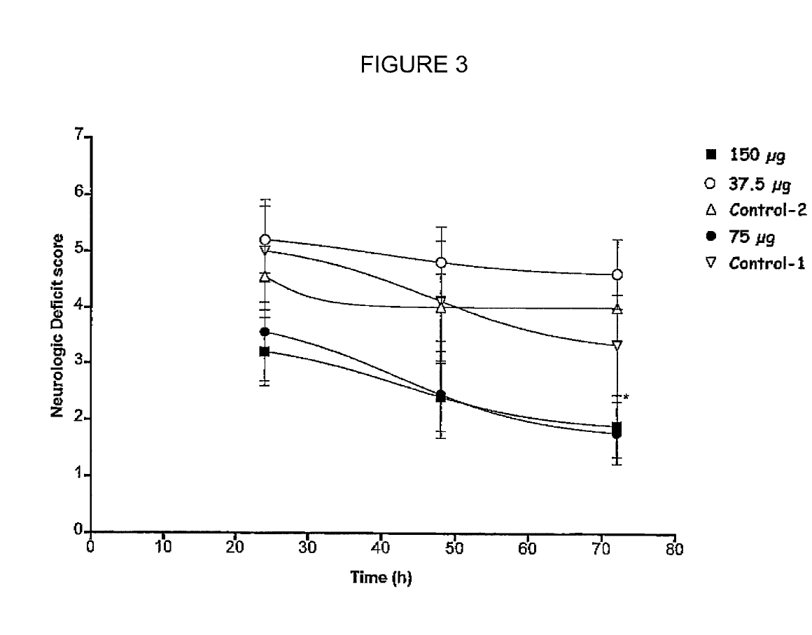

There was no significant difference in the placing test scores between the 75 μg rh-IGF-I and control-1 groups (p=0.187). There was a significant change in scores over time (p=0.001), and the change over time was not significantly different between the two groups (p=0.962). There were no significant differences between the 75 μg rh-IGFI and control-i groups at 4, 24, 48, or 72 hours. This shows that IN 150 μg IGF-I can also improve the sensorimotor function at 72 hours, and that the 75 μg rh-IGF-I treated group showed a recovery trend, but the difference did not reach statistical significance (FIG. 3).

Effect of IN IGF-I on Vestibulomotor Function Assessed by the Balance Beam Test

There was a significant difference in the balance beam test scores between the 150 μg, 37.5 μg, and control-2 groups (p=0.001) with the 150 μg rh-IGF-I group having lower scores on average than the 37.5 μg rh-IGF-I and control-2 groups. There was a significant change in scores over time (p=0.001), and the change over time was significantly different between the groups (p=0.047). At 48 hours, the average score in the 150 μg rh-IGF-I group was significantly lower than that in the 37.5 μg rh-IGF-I group (p=0.005). At 72 hours, the average score in the 150 μg rh-IGF-I group was significantly lower than that in the 37.5 μg rh-IGF-I and control-2 groups (p=0.001). There were no significant differences between the 150 μg, 37.5 μg, and control-2 groups at 4 hours and 24 hours.

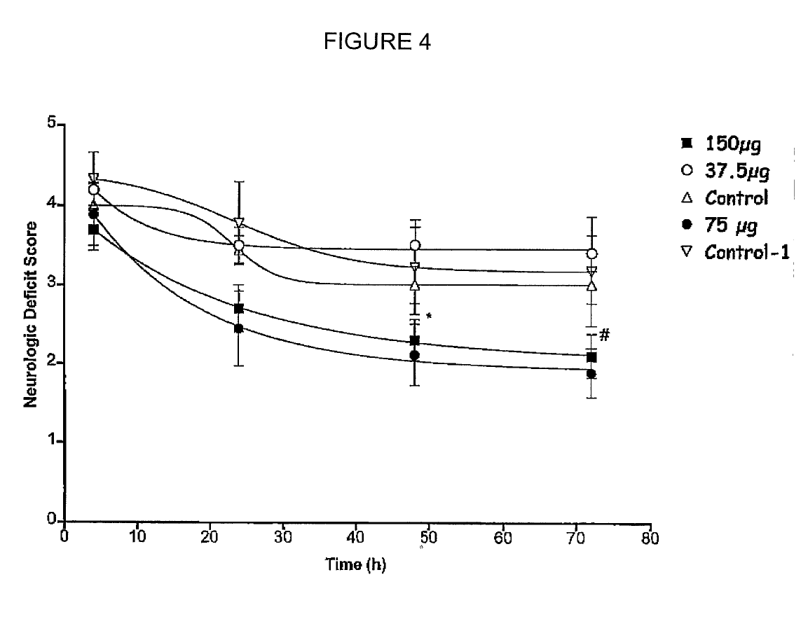

There was no significant difference in scores between the 75 μg rh-IGF-I and control-1 groups (p=0.114). There was a significant change in scores overtime (p=0.001), and the change over time was not significantly different between the groups (p=0.162). There were no significant differences between the 75 μg rh-IGF-I and control-I groups at 4, 24, 48 or 72 hours. This shows that 150 μg IGF-I was also effective at improving vestibulomotor function in rats with stroke (FIG. 4).

Effect of IN IGF-I on Somatosensory Function Assessed by the Adhesive Tape Test

The somatosensory function in the 150 and 75 μg rh-IGF-I treated groups showed a clear trend improvement, but only the 150 μg rh-IGF-I treated group reached statistical significance at 72 hours (remove time p=0.015; contact time p=0.012). The average remove scores at 72 hours were 2, 5, and 4 in the 150 μg, 37.5 μg, and control-2 groups, respectively. The average contact scores were 1, 5, and 4. The average remove and contact scores for the 75 μg rh-IGF-I and control-1 groups were 3 and 5, respectively. This function was affected only within 24 h in the ipsilateral forepaw (right), and then recovered to normal.

Correlation Between Behavioral Tests and Corrected Infarct Volume

In study 2, infarct volume was positively correlated with all of the neurologic behavioral test scores at 72 hours (p<0.001). In study 1, infarct volume was positively correlated with postural reflex and adhesive tape tests (p<0.01) but not significantly correlated with the forepaw placing and beam balance tests.

Body Weight Loss

There were no significant baseline body weight differences between the 150 μg rh-IGF-I, 37.5 μg rh-IGF-I, and control-2 groups (p=0.647) or between the 75 μg rhIGF-I and control-1 groups (p=0.460). The ANOVA also showed no significant body weight differences between the groups (study 2, p=0.339; study 1, p=0.675). There was a significant weight change over time (p=0.001). The change over time was not significantly different between the groups (study 2, p=0.293; study 1, p=0.721).

### Discussion

Three doses of IGF-I, 150 μg, 75 μg, and 37.5 μg, were tested at 10 minutes after the onset of MCAO in these studies. The 150 μg and 75 μg doses of IGF-I significantly reduced infarct volume similarly, while 150 μg was much better than 75 μg in improving neurologic function. The 37.5 μg dose was ineffective. Therefore, of the doses examined, 150 μg IGF-I administered IN gave the best results.

The four behavioral tests used to evaluate neurologic deficits in CNS functions including motor, sensory, reflex, and vestibulomotor systems were sensitive to the treatment effects of IN IGF-I. All these tests significantly correlated with infarct volume at 72 hours after the onset of MCAO. These tests are not complex and can be easily performed in the assessment of drug treatment effects using animal stroke models.

In conclusion, this is the first report demonstrating effective treatment of focal cerebral ischemic damage with IN IGF-I. IN IGF-I can effectively and rapidly reduce both infarct size and neurologic deficits in an animal stroke model. This noninvasive method of bypassing the BBB has multiple advantages compared with existing conventional methods. IGF-I can be preferentially delivered to the brain, reducing unwanted systemic effects. Furthermore, IN administration provides a simpler, safer, and more cost-effective method of delivery than other methods currently in use, such as ICV administration. IN administration of IGF-I and other therapeutic agents is promising for the treatment of stroke and other central nervous system disorders.

### EXAMPLE 2

### Window of Opportunity for Treatment of Focal Cerebral Ischemia with Intranasal Administration of IGF-I in Rats

The following experiments delineate the window of opportunity for treatment of focal cerebral ischemia using IN delivery of IGF-I at different times following MCAO in rats. Infarct size and neurologic deficit scores assessing motor, sensory, and vestibulomotor functions were used to evaluate the efficacy of IGF-I.

### Materials and Methods

Animal Preparation

A total of 69 adult male Sprague-Dawley rats, weighing 242-293 g, were anesthetized with 3% halothane for induction and 1.5% halothane in a mixture of air (80%) supplemented with oxygen (20%) for maintenance. Animal temperature was continuously monitored with a rectal probe and maintained at 37° C., with a heating pad at all times during surgical procedures.

Middle Cerebral Artery Occlusion

Focal brain ischemia of the right hemisphere was induced by the intraluminal suture MCAO method as previously described (Longa et al. (1989) Stroke 20:84-91; Chen et al. (1992) J. Cereb. Blood Flow Metab. 12:621-8). Briefly, the right common carotid artery (CCA), external carotid artery (ECA), and internal carotid artery (ICA) were exposed. A length (19±0.5 mm) of 4-0 monofilament nylon suture with its tip rounded by heating near a flame, was advanced from the ECA into the lumen of ICA until it blocked the origin of the middle cerebral artery (MCA). Animals were reanesthetized with halothane 140 minutes following MCAO and reperfusion was performed by withdrawal of the suture until the tip cleared the lumen of the ICA.

Intranasal Administration

Recombinant human IGF-I (rhIGF-I) and succinate-buffered vehicle (pH 6.0) were provided by Chiron Corporation (Emeryville, Calif.). Animals were respectively divided into IN IGF-I treated groups at 2 hours, 4 hours, or 6 hours after the onset of middle cerebral artery occlusion and the parallel vehicle control groups. The investigator was blinded as previously noted in Example 1. IN administration was performed essentially as described previously; see also Frey et al. (1997) Drug Delivery 4:87-92; Thorne et al. (1995) Brain Res. 692:278-283. A first dose of 40 μl treatment (150 μg rh IGF-I) or vehicle solution per rat were given in nose drops (5 μl per drop) over a 16-minute period, alternating drops every 2 minutes between the left and right nares. A second and third dose of treatment or vehicle solution were administered to each treated or control animal, respectively, 24 hours and 48 hours after the onset of MCAO. A total of three doses or vehicle were administered per rat.

Behavioral Testing

The behavioral neurologic deficits were assessed at 2 hours, and then for 7 days after the onset of MCAO. Three behavioral tests were used to systematically evaluate motor, sensory, and vestibulomotor deficits. Motor-sensory function was assessed by postural reflex test with a four-point scale (0-4) described by Bederson et al. (1986) Stroke 17:472-476 as noted in Example 1. Somatosensory deficits were measured by the modified adhesive tape test (Schallert et al. (1982) Pharmacol. Biochem. Behav. 16:445462; Herandez and Schaller (1988) Exp. Neurol. 102:318-324; Andersen et al. (1990) Physiol. Behav. 47:1045-1052). In the initial test, a small piece of adhesive-backed paper dot (113.1 mm²) was used as a tactile stimuli, occupying the ventral side of the left forepaw. The rat was then returned to its cage. Latencies to contact and removal from the paw were recorded for three trials per day. Individual trials were separated by at least 5 minutes. The average time of the three trials was used for each day's record. The animals were trained two days prior to surgery. Once the rats were able to remove the paper dots within 10 seconds, they were subjected to MCAO. The scale was evaluated as: 1=<10 seconds; 2=10-19 seconds; 3=20-29 seconds; 4=30-39 seconds; 5=40-49 seconds; 6=50-59 seconds; 7=>60 seconds. Vestibulomotor function was assessed by the modified beam balance test (Feeney et al. (1982) Science 217:855-857; Dixion et al. (1987) J. Neurosurg. 67:110-119) in which the animal was placed on a narrow beam (40×1.3×1.3 cm) for 60 seconds. Five training scores were recorded before MCAO. Functional deficit was evaluated on a five-point score: 1=steady posture with paws on top of the beam; 2=paws on side of the beam or wavering; 3=one or two limb(s) slip off; 4=three limbs slip off; 5=attempts with paws on the beam, but falls or drapes over the beam, then falls.

Histological Assessment

The rat brains were fixed by transcardial perfusion with normal saline, followed by neutral buffered 10% formalin under a deep halothane anesthesia at the 7-day point. Brains were removed, then cut into seven equally spaced coronal sections (2 mm thickness) from the frontal lobe to the occipital lobe. A series of adjacent 5-micrometer-thick sections embedded in paraffin were cut from each section in the coronal plane and stained with hematoxylin and eosin. An image analysis system (AIS/C, Imagine Research Inc., St. Catherines, Ontario, Canada) was used to measure the absolute infarct volume, and the right and left hemisphere volumes. The infarct size is presented as a percentage of the absolute infarct volume divided by the left hemisphere volume (Aspey et al. (1998) Neuropathol. Appl. Neurobiol. 24:487-497; Yao et al. (1999) Brain Res. 818:140-146).

Statistics

Statistical analyses were performed using the SAS 6.12 system. Mortality rates were analyzed by Fisher's exact test. Baseline body weight and infarct size were compared by t-tests. Behavioral tests and body weight changes were analyzed by repeated measure analysis of variance (ANOVA) models. Wilcoxon rank-sum tests were performed for each of the time points to see at what time point the scores became significantly different.

### Results

IN IGF-I administration initiated at 2 hours and 4 hours following MCAO significantly reduced infarct volume by 54% and 39%, respectively. The infarct size (19.7±5.0%, mean±SEM) in the IGF-I group was also lower than that of in the control group (29.1±4.5%) when treatment was delayed until at 6 hours following MCAO, but this did not reach statistical significance (p=0.18). Even with a delay of 6 hours, infarct volume was almost equal to the infarct size rats treated with IGF-I at 4 hours following MCAO (20±4.2% in IGF-I vs 33±4.0% in the control) (FIG. 1).

Motor-sensory function assessed by postural reflex test was significantly improved at 5, 6, and 7 days after IN IGF-I treatment initiated at 2 hours following MCAO. No significant improvement of this neurologic function was found when IN IGF-I was initiated at 4 hours or 6 hours after the onset of MCAO (FIG. 2).

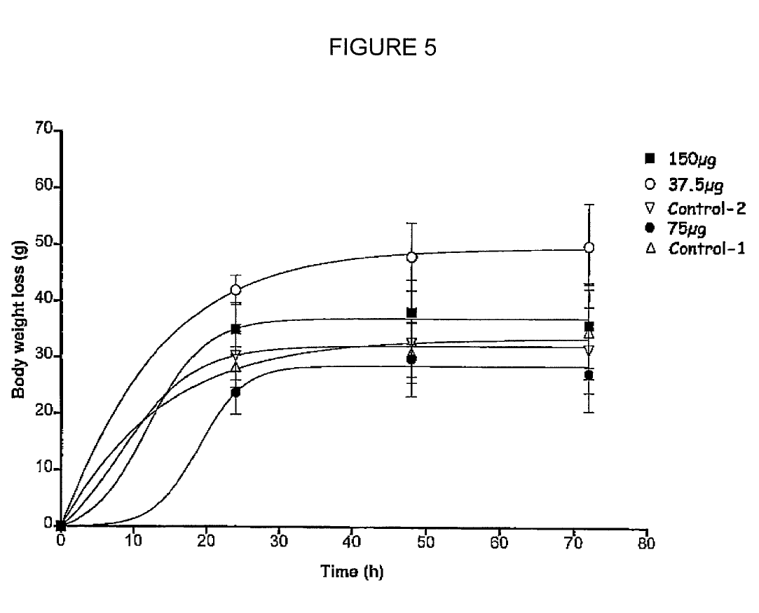

The change over time (from 1 to 7 days) of contact scores for the 2-hour treated group and the removal scores at 6-hour-treated group were significantly improved as compared with their vehicle controls, with a p-value of 0.02 and 0.04 respectively. There were no significant differences of contact and removal scores between the groups at 1-7 day time points (FIG. 3, 4). This showed that IGF-I still improved the somatosensory function assessed by the adhesive tape test even treatment was delayed until 6 hours after ischemia. There was no significant improvement of vestibulomotor function assessed by the balance beam test between the treated and control groups when treatment began at 2 h, 4 h, and 6 h following MCAO (FIG. 5).

Pearson correlation analyses showed that the infarct volumes were positively correlated with all of the three behavioral scores at the 7-day point in the 2 hour and 6 hour groups (p<0.01). Infarct volume was also correlated with the postural reflex test scores (p=0.03), but not correlated with the other two behavioral test scores at the 7-day point in the 4 hour groups.

Combining the 2 hour, 4 hour, and 6 hour treatment and control groups, overall mortality was lower in the IGF-I treated group (9%; 3/32) than that in the vehicle control (24%; 9/37), but did not reach statistical significance (p=0.10). There were no baseline body weight differences between the groups. Body weight change over the 7-day period was also not significantly different between the groups.

### Discussion

Intranasal administration of 150 μg IGF-I beginning at 2 hours after the onset of MCAO significantly reduces the infarct size and improves motor-sensory functions. Intranasal administration of IGF-I initiated at 4 hours following MCAO also reduces infarct volume. Even when IGF-I is delivered 6 hours after MCAO, there is a significant improvement of somatosensory function and a tendency to reduce infarct size.

In the clinical setting, many patients are not able to receive treatment within two hours after they have a stroke. The present study demonstrates that the therapeutic time window for effective treatment of IN delivery of IGF-I is prolonged at least to 6 hours following MCAO.

In conclusion, IN delivery of IGF-I provides a window of opportunity up to 6 hours after ischemia for treatment of brain damage and neurologic functional deficits in the MCAO model. IN delivery is a noninvasive and safer method of bypassing the blood-brain barrier than other methods such as ICV administration. Intranasally administered IGF-I is an excellent candidate for the treatment of ischemic events, as it reduces ischemic damage, including infarct size, edema, and neurologic deficit.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method for preventing or reducing ischemic damage in the central nervous system of a mammal, said method comprising administering intranasally (IN) a therapeutically effective amount of insulin-like growth factor-1 (IGF-I) or biologically active variant thereof to the central nervous system of said mammal, wherein said therapeutically effective amount comprises about 0.10 mg to about 3.0 mg per kg body weight of said mammal.

2. The method of claim 1, wherein said IGF-I is human IGF-I or biologically active variant thereof, wherein said variant retains IGF-I activity and has at least 70% sequence identity to the amino acid sequence for human IGF-I.

3. The method of claim 1, wherein said mammal is a human and wherein said IGF-I is human IGF-I.

4. The method of claim 1, wherein said ischemic damage is selected from the group consisting of a neurologic deficit, edema, and coagulation necrosis of a tissue of said central nervous system.

5. The method of claim 4, wherein said coagulation necrosis of said tissue results from death of cells selected from the group consisting of neuronal cells and glial cells.

6. The method of claim 1, wherein said ischemic damage is caused by stroke, multiple infarct dementia, cardiac arrest, shock, hypotension, perinatal asphyxia, traumatic injury to the central nervous system, or a cardiovascular surgical procedure.

7. The method of claim 6, wherein said ischemic damage is caused by stroke.

8. The method of claim 1, wherein said therapeutically effective amount comprises about 0.50 mg to about 2.0 mg per kg body weight of said mammal.

9. The method of claim 8, wherein said therapeutically effective amount comprises about 1.0 mg to about 1.5 mg per kg body weight of said mammal.

10. The method of claim 1, wherein said administering comprises applying said therapeutically effective amount of IGF-I or variant thereof to an upper one-third of a nasal cavity.

11. The method of claim 10, comprising applying said therapeutically effective amount of IGF-I or variant thereof to an olfactory area in said upper one-third of said nasal cavity.

12. The method of claim 10, comprising applying said therapeutically effective amount of IGF-I or variant thereof to a roof of a nose.

13. A method for treating a mammal following an ischemic event affecting the central nervous system of said mammal, said method comprising administering intranasally (IN) a therapeutically effective amount of insulin-like growth factor-I (IGF-I) or biologically active variant thereof to the central nervous system of said mammal, wherein said therapeutically effective amount comprises about 0.10 mg to about 3.0 mg per kg body weight of said mammal.

14. The method of claim 13, wherein said IGF-I is human IGF-I or biologically active variant thereof, wherein said variant retains IGF-I activity and has at least 70% sequence identity to the amino acid sequence for human IGF-I.

15. The method of claim 13, wherein said mammal is a human and wherein said IGF-I is human IGF-I.

16. The method of claim 13, wherein said ischemic event is selected from the group consisting of stroke, multiple infarct dementia, cardiac arrest, shock, hypotension, perinatal asphyxia, and traumatic injury to the central nervous system.

17. The method of claim 16, wherein said ischemic event is stroke.

18. The method of claim 13, wherein said therapeutically effective amount comprises about 0.50 mg to about 2.0 mg per kg body weight of said mammal.

19. The method of claim 18, wherein said therapeutically effective amount comprises about 1.0 mg to about 1.5 mg per kg body weight of said mammal.

20. The method of claim 13, wherein said administering comprises applying said therapeutically effective amount of IGF-I or variant thereof to an upper one-third of a nasal cavity.

21. The method of claim 20, comprising applying said therapeutically effective amount of IGF-I or variant thereof to an olfactory area in said upper one-third of said nasal cavity.

22. The method of claim 20, comprising applying said therapeutically effective amount of IGF-I or variant thereof to a roof of a nose.

23. The method of claim 13, wherein said treating results in a reduction in ischemic damage in the central nervous system of said mammal.

24. The method of claim 23, wherein said ischemic damage is edema.

25. The method of claim 23, wherein said ischemic damage is coagulation necrosis of a tissue in the central nervous system.

26. The method of claim 23, wherein said ischemic damage is a neurologic deficit.
